(19) United States
(12) Patent Application Publication
(10) Pub. No.: US 2010/0234673 A1
(43) Pub. Date: Sep. 16, 2010

(54) Suitable Apparatus for Precisely and Reproducibly Transferring a Fertilized Egg-Cell (Embryo) to the Uterus

(76) Inventor: Petrus Gerardus Alphonsus Hompes, Amstelveen (NL)

Correspondence Address: PEACOCK MYERS, P.C., 201 THIRD STREET, N.W., SUITE 1340, ALBUQUERQUE, NM, 87102, US

(21) Appl. No.: 12/594,188

(22) PCT Filed: Feb. 27, 2008
(86) PCT No.: PCT/NL08/50116

§ 371 Date: Mar. 23, 2010

(30) Foreign Application Priority Data
Mar. 8, 2007 (NL) 2000528

(51) Int. Cl. A61B 17/43

(52) U.S. Cl. 600/35

(57) ABSTRACT

The subject of the present invention is in vitro fertilisation (IVF) and intracytoplasmic sperm injection (ICSI), and relates to an apparatus with which embryo transfer can be standardised and which, in comparison with the currently used technique, may possibly result in higher pregnancy rates. This is realised by using an apparatus with which it is possible to precisely and reproducibly aspirate one or two embryos from a Petri dish and to transfer them to the uterus, to a particular distance from the fundus. It has been shown that the chance of pregnancy is best if the distance between the embryo and the fundus is 1-2 cm.

The present invention relates to a suitable apparatus for precisely and reproducibly transferring an outside the uterus fertilized egg-cell (embryo) to the uterus.

It is known that a considerable number of women are unable to become pregnant. The cause for this may rest with the man as well as with the woman.

Many of these women have a strong desire to have children and take recourse to artificial fertilization techniques such as in vitro fertilization (IVF) and intracytoplasmic sperm injection (ICSI).

The IVF/ICSI treatment involves several phases. The first phase concerns stimulation, wherein the patient receives hormones in order to bring a number of egg-cells to maturation. This is monitored by ultrasound.

The second phase involves puncturing, whereby the various follicles are pierced with the aid of a vaginal ultrasound probe, after which their contents are aspirated. The aspirated fluid is then passed on to the embryologist, who first establishes whether the fluid contains any egg-cells. If egg-cells are present, semen will be added in vitro to effect fertilization. After 2-3 days, the egg-cells are examined for cell division, whereby several fertilized egg-cells, also referred to as embryos, may be present.

In the third phase, the obtained fertilized egg-cells, embryos, are transferred to the uterus.

According to the prior art, this transfer takes place with the aid of a syringe attached to a catheter. By means of the syringe the embryos are aspirated with the fluid and with the aid of ultrasound guiding they are transferred to the uterus. The woman may decide whether one or two embryos are transferred to the uterus, with the chance of one or two children. For this purpose a standard commercially available 1 ml syringe is used. The catheter is comprised of a thin, flexible plastic tube, in which the embryos are enclosed between two air bubbles.

In an endeavour to improve the known method so as to increase the chance of a pregnancy much more attention is given to improving stimulation protocols than to improving the embryo transfer technique.

However, various studies have shown that the chance of pregnancy resulting from IVF/ICSI is influenced very particularly by the location to which the embryo is transferred in relation to the fundus of the uterus. It has been shown that the best chance of pregnancy is afforded by a distance of 1-2 cm between the transferred embryo and the fundus (see for example M. Lambers et al. ‘The position of the transfer air bubbles after embryo transfer is related to pregnancy rates’, accepted for publication in Fertility and Sterility 2006).

However, the prior art technique is imprecise and not reproducible, making it difficult to determine the transfer position of the embryo. The imprecision and non-reproducibility are caused by a number of factors such as the syringe itself, the resistance of the plunger, the pressure exerted on the plunger by the physician, and possible intra-uterine resistance partly attributable to uterine contractions. Especially the varying pressures applied when ejecting the embryo from the catheter plays a considerable role in the above mentioned imprecision and non-reproducibility.

Moreover, excessive ejection pressure may damage the embryos themselves.

It is thus an object of the invention to provide an apparatus, which effectively eliminates the above mentioned drawbacks, and is able to significantly increase the chance of pregnancy resulting from an IVF treatment.

To this end, the present invention provides a suitable apparatus for precisely and reproducibly transferring to the uterus an egg-cell (an embryo) fertilized outside the uterus, which apparatus is characterised in that the same is provided with a pump, a flexible tube, a motor, control electronics, a power supply and a catheter.

Surprisingly, it was shown that by using the apparatus according to the invention, the drawbacks of the prior art are effectively removed. A special feature of the electrical pump used for embryo transfer is that it is able to aspirate a very small amount of fluid (approximately 30 microlitres) within an exact time limit, as well as to inject this small amount in a reproducible manner.

The person in question, usually a physician who has to transfer the embryo to the uterus, is able to do this with the aid of the present apparatus precisely and reproducibly, such that the embryo is placed in the uterus at a proven optimal distance, preferably at 1-2 cm from the fundus.

In accordance with the present apparatus, first an amount of IVF culture medium is aspirated into the catheter, then an air bubble, and then again an amount of IVF culture medium containing one or several embryo(s), then again an air bubble, and finally another amount of culture medium. The embryos are then, as it were, enclosed in the catheter between the two air bubbles.

With the aid of ultrasound guiding the treating physician then inserts the catheter into the uterus and ensures that the end of the catheter is positioned at the desired location, preferably at 1-2 cm from the fundus of the uterus. Then the pump is switched on and the embryos are ejected slowly and evenly from the catheter.

When the embryos have being placed into the uterus at the desired distance from the fundus, the catheter is withdrawn from the uterus.

The control electronics provided in the apparatus enable the suction and pushing force of the apparatus according to the invention to be adjusted with precision. In comparison with the prior art syringe, it is possible that the chance of pregnancy may be significantly increased with the aid of the apparatus according to the invention.

An important advantage of the present apparatus is further that diverse physicians are able to use it with the same precision and reproducibility because when ejecting the embryos from the catheter using the pump, the pressure can be kept constant, which is not possible with the syringe. The difference to using a syringe is that the ejection pressure may vary depending on the person operating the syringe.

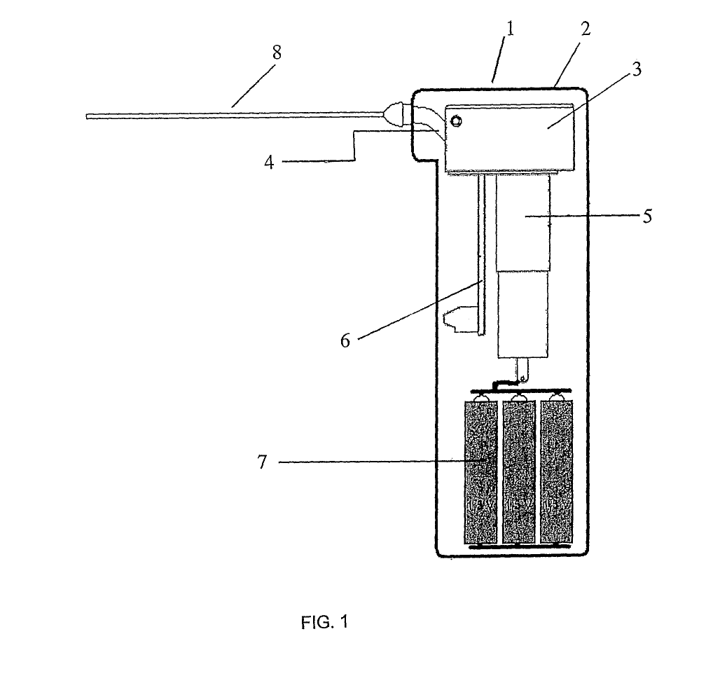

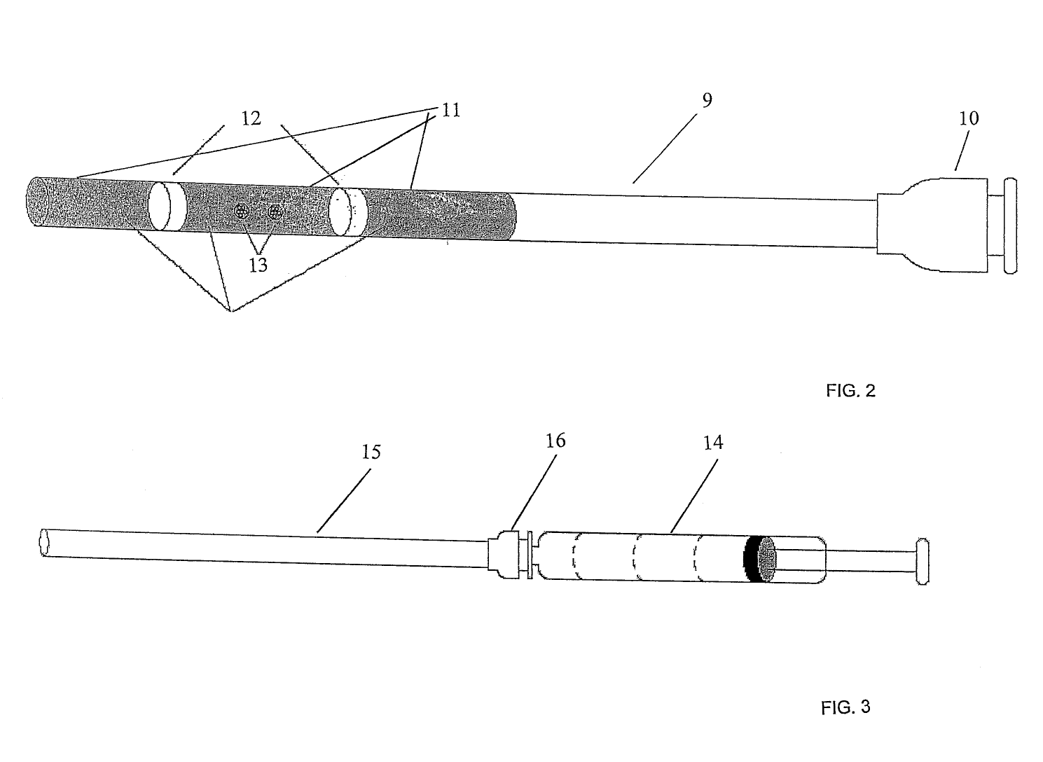

The invention will now be further explained with reference to the accompanying FIGS. 1-3. The invention is not limited to the embodiments illustrated in the FIGS. 1 and 2.

FIG. 1 shows the apparatus according to the invention.

FIG. 2 shows the catheter according to the invention, and

FIG. 3 shows the apparatus according to the prior art.

FIG. 1 shows the apparatus (1) according to the invention, provided with a pump (3), a flexible tube (4), a motor (5), control electronics (6), a power supply (7) and catheter (8). It is usually that a battery serves as power supply, but the mains power system is also possible, in which case the apparatus is provided with a cord and plug.

The pump (3), flexible tube (4), motor (5), control electronics (6) and power supply (7) are preferably accommodated in a housing (2) for easy handling of the apparatus (1) and for the protection of the various components in the apparatus (1).

Via the catheter, the embryologist will with the aid of the apparatus first aspirate in vitro a small amount of culture medium, then an air bubble, then an amount of culture medium containing one or two embryos, depending on the wish of the patient, then air again and finally again an amount of culture medium. In total, the amount of inserted fluid is only 30 microlitres.

The treating physician, usually a gynaecologist, will subsequently take the apparatus to the treatment room where the transfer of the embryo takes place.

With the aid of ultrasound guiding the treating surgeon will insert the catheter into the uterus such that its end is located at a distance of 1-2 cm from the fundus. Using the present apparatus, the contents of the catheter are then gradually, at a constant pressure ejected from the catheter such that the air bubbles, and thus the embryos, are deposited at a distance of 1-2 cm from the fundus of the uterus. Any arbitrary physician is able to carry out this operation consistently and reproducibly, with the possible result of the thus treated woman having a significantly greater chance of pregnancy.

In the apparatus (1) according to the invention, the power supply (7) drives the motor (5) bringing about the pumping action of the pump (3). Instead of batteries, it is possible to use another power source, for example the electric power mains, in which case batteries are superfluous. The suction and ejection pressure generated by the pump (3) is passed on via catheter (8), which by means of the flexible tube (4) is connected with the pump (3). It is preferred for the catheter to be removably connected with the flexible tube (4).

FIG. 2 shows a catheter (9), whose one end is provided with a connecting piece (10) for connecting to the end of the flexible tube (4).

In FIG. 2, the amount of culture medium is indicated with reference numeral (11), the air bubbles carry reference numeral (12), and the embryo(s) reference numeral (13).

The apparatus according to the prior art is represented in FIG. 3. In FIG. 3, the catheter (15) is connected with a syringe (14) by means of a connection (16).

With regard to precision and reproducibility, this apparatus is inferior to the apparatus according to the invention, as a consequence of which the chance of pregnancy when using such an apparatus is significantly smaller than when using the present apparatus.

It should be noted that the present invention is not limited to the embodiments shown in the FIGS. 1 and 2 of the present invention.

1. Apparatus (1) suitable for precisely and reproducibly transferring an outside the uterus fertilized egg-cell (embryo) to the uterus, which apparatus (1) is provided with a pump (3), a flexible tube (4), a motor (5), control electronics (6), a power supply (7) and a catheter (8), characterised in that the pump (3), the flexible tube (4), the motor (5), the control electronics (6), and the power supply (7) are accommodated in a housing (2).

2. An apparatus (1) according to claim 1, wherein the power supply (7) is a battery.

3. An apparatus (1) according to claim 1 or 2, characterised in that the catheter (8) is removably connected with the flexible tube (4).
